(19) United States
(12) Patent Application Publication
(10) Pub. No.: US 2023/0190473 A1
(43) Pub. Date: Jun. 22, 2023

(54) ELECTRONIC IMPLANTABLE PENILE PROSTHESIS WITH PRESSURE REGULATION AND OTHER FUNCTIONS

(71) Applicant: Boston Scientific Scimed, Inc., Maple Grove, MN (US)

(72) Inventors: Noel Smith, Windgap (IE); Eduardo Marcos Larangeira, Cork City (IE); Brian P. Watschke, Minneapolis, MN (US); Daragh Nolan, Youghal (IE); Thomas Sinnott, Wexford (IE); Evania Ann Mareena, Clonmel (IE); John Gildea, Kilcock (IE)

(21) Appl. No.: 18/068,108

(22) Filed: Dec. 19, 2022

Related U.S. Application Data
(60) Provisional application No. 63/265,812, filed on Dec. 21, 2021.

(51) Int. Cl. A61F 2/26; A61F 2/48

(52) U.S. Cl. CPC A61F 2/26; A61F 2/482; A61F 2/484; A61F 2250/0013

(57) ABSTRACT

According to an aspect, an inflatable penile prosthesis includes a fluid reservoir configured to hold fluid, an inflatable member, and an electronic pump assembly configured to transfer the fluid between the fluid reservoir and the inflatable member. The electronic pump assembly includes a pump, an active valve, a pressure sensor configured to measure a pressure of the inflatable member, and a controller configured to control at least one of the pump or the active valve based on the measured pressure.

### CROSS-REFERENCE TO RELATED APPLICATION

This application claims priority to U.S. Provisional Patent Application No. 63/265,812, filed on Dec. 21, 2021, entitled “ELECTRONIC IMPLANTABLE PENILE PROSTHESIS WITH PRESSURE REGULATION AND OTHER FUNCTIONS”, the disclosure of which is incorporated by reference herein in its entirety.

### TECHNICAL FIELD

This disclosure relates generally to bodily implants and more specifically to bodily implants, such as an electronic implantable penile prosthesis with pressure regulation and other functions.

### BACKGROUND

One treatment for male erectile dysfunction is the implantation of a penile prosthesis that erects the penis. Some existing penile prosthesis include inflatable cylinders or members that can be inflated or deflated using a pump mechanism. The pump mechanism includes a pump, implantable in the scrotum, that can be manually squeezed by the user to move fluid from a reservoir into the cylinders, creating an erection. For some patients, the manual pumping procedure may be relatively challenging.

### SUMMARY

According to an aspect, an inflatable penile prosthesis includes a fluid reservoir configured to hold fluid, an inflatable member, and an electronic pump assembly configured to transfer the fluid between the fluid reservoir and the inflatable member. The electronic pump assembly includes a pump, an active valve, a pressure sensor configured to measure a pressure of the inflatable member, and a controller configured to control at least one of the pump or the active valve based on the measured pressure.

According to some aspects, the inflatable penile prosthesis may include one or more of the following features (or any combination thereof). The controller is configured to cause the active valve to be in an open position to transfer a portion of the fluid from the inflatable member to the fluid reservoir in response to the measured pressure being greater than a threshold. The controller is configured to cause the pump to operate to transfer a portion of the fluid from the fluid reservoir to the inflatable member in response to the measured pressure being less than a threshold. The fluid reservoir includes a pressure-regulating flexible member configured to cause the inflatable member to be partially inflated without operating the pump. The electronic pump assembly includes an accelerometer configured to measure an acceleration of a user of the inflatable penile prosthesis. The controller is configured to identify a sleep pattern of a user of the inflatable penile prosthesis based on the measured acceleration, and the controller configured to cause the inflatable member to at least partially inflate when the user is asleep. The electronic pump assembly includes a heart rate sensor configured to monitor a heart rate of a user of the inflatable penile prosthesis. The electronic pump assembly includes a temperature sensor configured to measure a temperature of the fluid. The controller is configured to iteratively initiate an inflation cycle and a deflation cycle during a period of time, wherein at each subsequent iteration, the pressure in the inflatable member is increased.

According to an aspect, an inflatable penile prosthesis includes a fluid reservoir configured to hold fluid, an inflatable member, and an electronic pump assembly configured to transfer the fluid between the fluid reservoir and the inflatable member. The electronic pump assembly includes an antenna configured to receive a wireless signal from an external device, a pump, an active valve, a pressure sensor configured to measure a pressure of the inflatable member, and a controller configured to control at least one of the pump or the active valve based on at least one of the measured pressure or the wireless signal.

According to some aspects, the inflatable penile prosthesis may include one or more of the following features (or any combination thereof). The electronic pump assembly includes an accelerometer configured to measure an acceleration of a user of the inflatable penile prosthesis. The controller is configured to determine a type of activity based on the measured acceleration, where the controller is configured to adjust a pressure sensing rate associated with the pressure sensor based on the type of activity. The controller is configured to partially inflate the inflatable member such that the pressure of the inflatable member does not exceed a threshold, where, in response to the wireless signal, the controller is configured to inflate the inflatable member up to a maximum pressure threshold during an inflation cycle. The electronic pump assembly includes a temperature sensor configured to measure a temperature of the fluid, where, in response to the measured temperature being greater than a threshold, the controller is configured to transmit, over a network, a notification message via the antenna to one or more external devices. The controller includes a memory configured to store at least one of a maximum pressure threshold or a partial pressure threshold, where the controller is configured to update a value of at least one of the maximum pressure threshold or the partial pressure threshold based on information received from the external device via the antenna. The controller is configured to detect a performance issue associated with the inflatable penile prosthesis based on pressure readings from the pressure sensor, where the controller is configured to transmit, over a network, a notification message to one or more external devices in response to the performance issue being detected. The electronic pump assembly includes a battery configured to power the controller, the electronic pump assembly including a sensor configured to monitor a performance of the battery. The controller is configured to obtain pressure readings from the pressure sensor over time according to a pressure sensor rate, where the controller is configured to detect a first pressure pulsation at a first time and a second pressure pulsation at a second time, and the controller is configured to cause the active valve to be in an open position after the first time and configured to cause the active valve to be in a closed position before the second time. The electronic pump assembly includes a check valve in series with the pump.

According to an aspect, a method of operating an inflatable penile prosthesis include receiving, by an antenna of an electronic pump assembly, a wireless control signal from an external device, activating, by a controller, a pump of the electronic pump assembly to operate to transfer fluid from a fluid reservoir to an inflatable member in response to the wireless control signal such that a pressure of the inflatable member reaches a threshold, measuring, by a pressure sensor, the pressure of the inflatable member, and activating, by the controller, an active valve of the electronic pump assembly to be in an open position to transfer a portion of the fluid from the inflatable member to the fluid reservoir in response to the measured pressure being greater than the threshold. In some examples, the method includes detecting, by the controller, that the measured pressure corresponds to the threshold. In some examples, the method includes activating, by the controller, the active valve to be in a closed position in response to the measured pressure being detected as corresponding to the threshold.

### BRIEF DESCRIPTION OF THE DRAWINGS

FIG. 1A illustrates an inflatable penile prosthesis having an electronic pump assembly according to an aspect.

FIG. 1B illustrates a controller of the electronic pump assembly according to an aspect.

FIG. 2A illustrates an inflatable penile prosthesis having an electronic pump assembly according to another aspect.

FIG. 2B illustrates a check valve in series with a pump of the electronic pump assembly according to an aspect.

FIG. 2C illustrates a check valve in series with a pump of the electronic pump assembly according to another aspect.

FIG. 3 illustrates an example of an electronic pump assembly according to an aspect.

FIG. 4 illustrates an example of an electronic pump assembly according to another aspect.

FIG. 5 illustrates an inflatable penile prosthesis having an electronic pump assembly according to another aspect.

FIG. 6 illustrates a flow chart depicting example operations of an electronic pump assembly according to an aspect.

### DETAILED DESCRIPTION

This disclosure relates to an inflatable penile prosthesis that includes an electronic pump assembly to transfer fluid between a fluid reservoir and an inflatable member. The electronic pump assembly may wirelessly communicate with an external device (e.g., a computer, a smartphone, tablet, pendant, key fob, etc.) to control the inflatable penile prosthesis (e.g., inflate or deflate the inflatable member, update one or more control parameters). The electronic pump assembly may include a primary battery (e.g., a non-rechargeable battery) or a rechargeable battery configured to be recharged by an external charger.

The electronic pump assembly includes one or more pumps (e.g., electronically-controlled pumps such as one or more electromagnetic or Piezoelectric pumps), one or more active valves, and a controller. In some examples, the electronic pump assembly includes an accelerometer, a heart rate monitor, and one or more sensors to monitor a battery or other electronics on the electronic pump assembly. In some examples, the electronic pump assembly includes a temperature sensor.

The controller may actuate the pump(s) and the active valve(s) to control the inflation and deflation of the inflatable member based on control signals transmitted to the pump(s) and the active valve(s). The pump(s) may be unidirectional or bidirectional. In some examples, the electronic pump assembly includes one or more pumps in parallel with an active valve. In some examples, the pump(s) can transfer the fluid to the inflatable member during an inflation cycle, and the active valve may transition to an open position to permit fluid to transfer back to the fluid reservoir during a deflation cycle. The pump(s) may transfer fluid on demand to the inflatable member at a relatively high-pressure rate. In some examples, the electronic pump assembly includes two or more parallel pumps such as a first pump and a second pump, where the first pump and the second pump are configured to operate out of phase from each other, which can increase the efficiency of the pumping operations. In some examples, the use of parallel pumps that operate out of phase from each other may allow the pumps to operate at lower frequencies, which can reduce power and improve battery life. In some examples, the electronic pump assembly may include one or more pumps in series with a pump, which can increase the amount of fluid that can be transferred to the inflatable member during a period of time. In some examples, the electronic pump assembly includes one or more pumps (e.g., emptying pumps) in series with the active valve. In some examples, the electronic pump assembly may include a check valve in series with one or more pumps (e.g., filling pumps).

An individual pump may include one or more passive check valves which transition to a closed position in response to positive pressure between the inflatable member and the fluid reservoir. In some examples, the active valve may transition to a closed position to hold (e.g., substantially hold) the pressure in the inflatable member. In some examples, the active valve may transition to an open position to release pressure in the inflatable member and/or allow a flowback to the inflatable member. In some examples, the electronic pump assembly includes a single active valve. In some examples, the electronic pump assembly includes multiple active valves. For example, one or more active valves may be in series with a pump or in parallel with a pump.

The electronic pump assembly may include a pressure sensor configured to sense a pressure of the inflatable penile prosthesis. In some examples, the pressure sensor is coupled to the inflatable member. The pressure sensor may measure the pressure in the inflatable member. The controller may receive the measured pressure from the pressure sensor and automatically control the active valve(s) and/or pump(s) to regulate the pressure in the inflatable member.

For example, the electronic pump assembly may receive a wireless signal from the external device. The wireless signal may cause the controller to initiate an inflation cycle to transfer fluid from the fluid reservoir to the inflatable member such that a pressure of the inflatable member reaches a threshold. To initiate the inflation cycle, the controller may activate the active valve to be in a closed position and activate a pump (or multiple pumps) to move the fluid to the inflatable member until the pressure of the inflatable member reaches the threshold.

The controller and the pressure sensor may regulate the pressure of the inflatable member (e.g., during sexual activity). For example, in the event of compression of the inflatable member, a pressure increase (or spike) may occur. However, the pressure regulation discussed herein may minimize or prevent injury to the patient or damage to the device. For example, the controller may receive pressure readings from the pressure sensor, and, in response to the measured pressure being greater than the threshold (or greater than the threshold exceeding a period of time), the controller may cause the active valve to be in the open position to transfer a portion of the fluid from the inflatable member back to the fluid reservoir. In response to the measured pressure corresponding to the threshold (or less than the threshold), the controller may switch the active valve to be in the closed position.

Also, there may be a small amount of leakage through the pump(s), the passive valve(s), and the active valve(s), and this leakage may increase at greater pressure escalations during sexual activity. However, in response to the measured pressure being less than a target threshold, the controller may activate the pump(s) to transfer additional fluid to the inflatable member such that the target pressure can be achieved.

In some examples, when the inflatable member is inflated to its target pressure (e.g., during sexual activity), the controller may increase the pressure sensing rate at which the controller receives pressure readings (e.g., the pressure is measured at high-speed intervals during sexual activity). When the inflatable member is not inflated to its target pressure, the controller may decrease the pressuring sensing rate, so that power and battery energy may be saved. These pressure regulating operations and other enhanced operations of the inflatable penile prosthesis are further explained with reference to the figures.

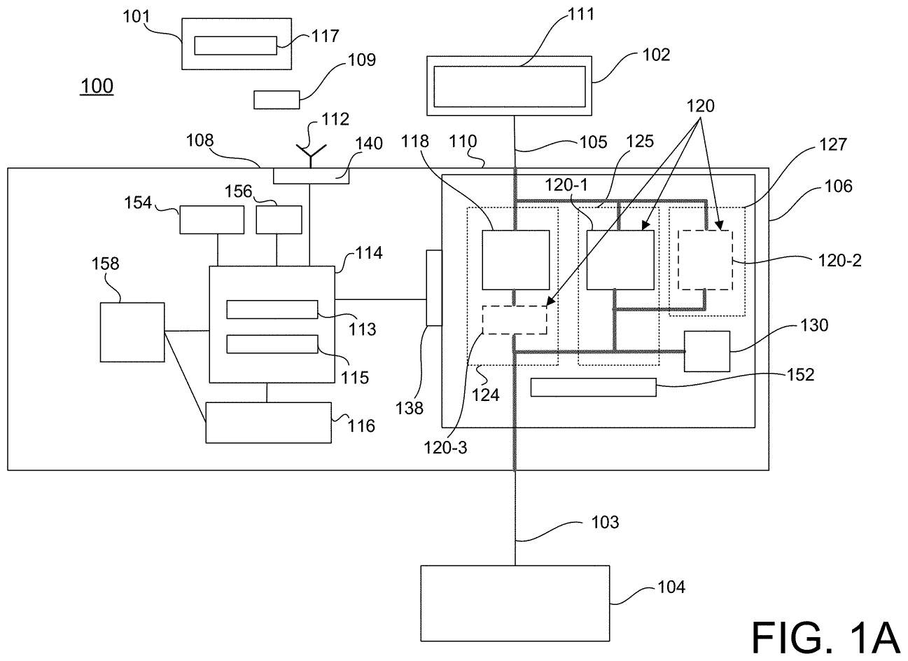

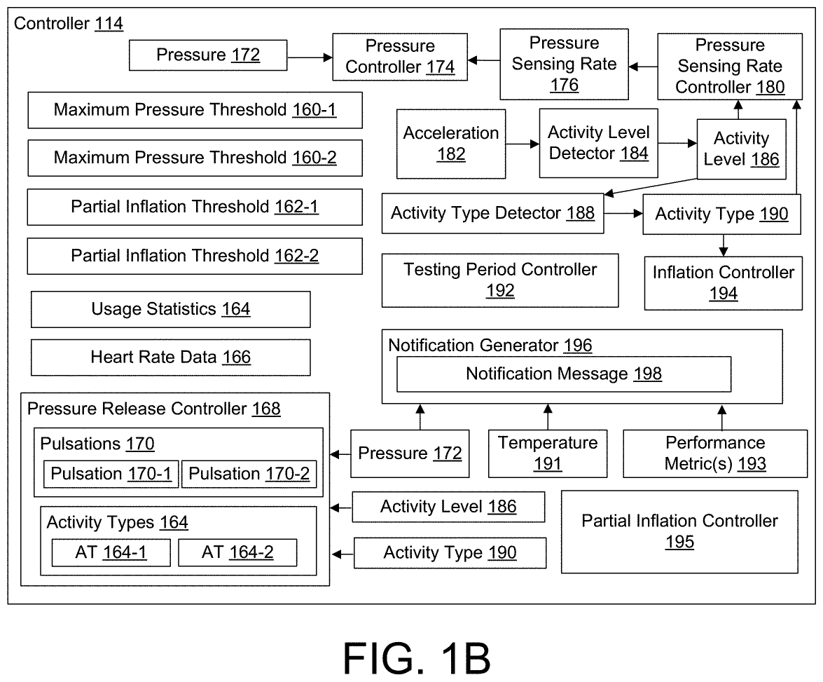

FIG. 1A illustrates an inflatable penile prosthesis 100 having an electronic pump assembly 106 that can improve the performance of the inflatable penile prosthesis 100 according to an aspect. FIG. 1B illustrates an example of a controller 114 of the electronic pump assembly 106 according to an aspect. The inflatable penile prosthesis 100 includes a fluid reservoir 102, an inflatable member 104, and an electronic pump assembly 106 configured to transfer fluid between the fluid reservoir 102 and the inflatable member 104. The inflatable member 104 may be implanted into the corpus cavernosum of the user, and the fluid reservoir 102 may be implanted in the abdomen or pelvic cavity of the user (e.g., the fluid reservoir 102 may be implanted in the lower portion of the user's abdominal cavity or the upper portion of the user's pelvic cavity). In some examples, at least a portion of the electronic pump assembly 106 may be implemented in the patient's body.

The inflatable member 104 may be capable of expanding upon the injection of fluid into a cavity of the inflatable member 104. For instance, upon injection of the fluid into the inflatable member 104, the inflatable member 104 may increase its length and/or width, as well as increase its rigidity. In some examples, the inflatable member 104 includes a pair of inflatable cylinders or at least two cylinders, e.g., a first cylinder member and a second cylinder member. The volumetric capacity of the inflatable member 104 may depend on the size of the inflatable cylinders. The volume of fluid in each cylinder may vary from about 10 milliliters in smaller cylinders and to about 50 milliliters in larger sizes. In some examples, the first cylinder member may be larger than the second cylinder member. In some examples, the first cylinder member may have the same size as the second cylinder member.

The fluid reservoir 102 may include a container having an internal chamber configured to hold or house fluid that is used to inflate the inflatable member 104. The volumetric capacity of the fluid reservoir 102 may vary depending on the size of the inflatable penile prosthesis 100. The volumetric capacity of the fluid reservoir 102 may be 3 to 150 cubic centimeters. In some examples, the fluid reservoir 102 is constructed from the same material as the inflatable member 104. In some examples, the fluid reservoir 102 is constructed from a different material than the inflatable member 104. In some examples, the fluid reservoir 102 contains a larger volume of fluid than the inflatable member 104.

In some examples, the fluid reservoir 102 is (or includes) a pressure-regulating flexible member 111. The pressure-regulating flexible member 111 may cause the inflatable member 104 to be partially inflated without activating any of the pumps 120. In some examples, the pressure-regulating flexible member 111 includes an expandable balloon. By providing a pressure-regulating flexible member 111 with a pounds per square inch (PSI) pressure level, some of the fluid may transfer from the fluid reservoir 102 to the inflatable member. In some examples, the PSI pressure level is in a range of 1.0 PSI to 5.0 PSI. In some examples, the PSI pressure level is in a range of 2.0 PSI to 4.0 PSI. In some examples, the PSI pressure level is (or around) 3.0 PSI. During a preliminary period (e.g., before the start of the inflation cycle), the pressure of the pressure-regulating flexible member 111 may cause the transfer of fluid from the fluid reservoir 102 to the inflatable member 104. In some examples, the fluid reservoir 102 is the expandable balloon. In some examples, the pressure-regulating flexible member 111 is a structure separate from the fluid reservoir 102 but disposed inside the cavity of the fluid reservoir 102. In some examples, the pressure-regulating flexible member 111 includes an expandable balloon disposed inside the cavity of the fluid reservoir 102.

The inflatable penile prosthesis 100 may include a first conduit connector 103 and a second conduit connector 105. Each of the first conduit connector 103 and the second conduit connector 105 may define a lumen configured to transfer the fluid to and from the electronic pump assembly 106. The first conduit connector 103 may be coupled to the electronic pump assembly 106 and the fluid reservoir 102 such that fluid can be transferred between the electronic pump assembly 106 and the fluid reservoir 102 via the first conduit connector 103. For example, the first conduit connector 103 may define a first lumen configured to transfer fluid between the electronic pump assembly 106 and the fluid reservoir 102. The first conduit connector 103 may include a single or multiple tube members for transferring the fluid between the electronic pump assembly 106 and the fluid reservoir 102.

The second conduit connector 105 may be coupled to the electronic pump assembly 106 and the inflatable member 104 such that fluid can be transferred between the electronic pump assembly 106 and the inflatable member 104 via the second conduit connector 105. For example, the second conduit connector 105 may define a second lumen configured to transfer fluid between the electronic pump assembly 106 and the inflatable member 104. The second conduit connector 105 may include a single or multiple tube members for transferring the fluid between the electronic pump assembly 106 and the inflatable member 104. In some examples, the first conduit connector 103 and the second conduit connector 105 may include a silicone rubber material. In some examples, the electronic pump assembly 106 may be directly connected to the fluid reservoir 102.

The electronic pump assembly 106 may automatically transfer fluid between the fluid reservoir 102 and the inflatable member 104 without the user manually operating a pump (e.g., squeezing and releasing a pump bulb). The electronic pump assembly 106 includes one or more pumps 120, one or more active valves 118, a controller 114 configured to control the pump(s) 120 and the active valve 118, and one or more pressure sensors 130. For example, the controller 114 may control the pump(s) 120 to pump fluid between the fluid reservoir 102 and the inflatable member 104. The controller 114 may control the active valve 118 to transition between an open position and a closed position. The pump(s) 120 is configured to transfer fluid (on demand) to the inflatable member 104 at relatively high-pressure (e.g., up to approximately 20.0 PSI).

The electronic pump assembly 106 includes a battery 116 configured to provide power to the controller 114 and other components on the electronic pump assembly 106. In some examples, the battery 116 is a non-rechargeable battery. In some examples, the battery 116 is a rechargeable battery. In some examples, the electronic pump assembly 106 (or a portion thereof) (or the controller 114) is configured to be connected to an external charger to charge the battery 116. In some examples, the electronic pump assembly 106 defines a charging interface that is configured to connect to (or placed in proximity to) the external charger. In some examples, the charging interface includes a universal serial bus (USB) interface configured to receive a USB charger. In some examples, the charging technology is electromagnetic or Piezoelectric.

The electronic pump assembly 106 includes an antenna 112 configured to wirelessly transmit (and receive) wireless signals 109 to (and from) an external device 101 (or multiple external devices 101). The external device 101 may be any type of component that can communicate with the electronic pump assembly 106. The external device 101 may be a computer, smartphone, tablet, pendant, key fob, etc. In examples, the external device 101 includes one or more devices associated with the user of the inflatable penile prosthesis and one or more devices associated with a physician. In some examples, the external device 101 includes a server computer configured to receive data over a network. In some examples, the external device 101 includes an application 117 executable by an operating system of the external device 101 or by a browser of the external device 101. The application 117 may define one or more user interfaces that permit a user to control the inflatable penile prosthesis 100 and set (or update) more settings or control parameters associated with the inflatable penile prosthesis 100.

A user may use the external device 101 to control the inflatable penile prosthesis 100. The user may use the external device 101 to inflate or deflate the inflatable member 104. For example, in response to the user activating an inflation cycle using the external device 101 (e.g., selecting a user control on the external device 101), the external device 101 may transmit a wireless signal 109 to the electronic pump assembly 106 to initiate the inflation cycle (received via the antenna 112), where the controller 114 may control the active valve(s) 118 and the pump(s) 120 to inflate the inflatable member 104 to a target inflation pressure, which can be up to a maximum pressure threshold 160-1 (defined by the physician) or a maximum pressure threshold 160-2 (defined by the patient). The controller 114 may cause the active valve to a closed position and activate the pump(s) to move fluid from the fluid reservoir 102 to the inflatable member 104. The controller 114 may actuate the pump(s) 120 according to a pump frequency. In some examples, the pumping architecture is designed such that audio frequencies are minimized or avoided. The frequency range of 50 hz to 19 khz can be perceived by humans. The controller 114 may actuate a pump 120 according to a pump frequency of 50 hz or less. In some examples, the controller 114 actuates a pump 120 according to a pump frequency of 40 hz or less. In some examples, the controller 114 actuates a pump 120 according to a pump frequency of 30 hz or less.

The maximum pressure threshold 160-1 may be defined by the physician and is the maximum allowable inflation pressure of the inflatable member 104. In some examples, the maximum pressure threshold 160-1 is programmed at the controller 114 (and can be adjusted by using an external device 101 to update the value stored at the controller 114). The maximum pressure threshold 160-2 may be defined by the patient and is less than (or within the range of) the maximum pressure threshold 160-1. For example, if the maximum pressure threshold 160-1 is 20.0 PSI, the maximum pressure threshold 160-2 can be 20.0 PSI or less (but not greater than 20.0 PSI). In some examples, the maximum pressure threshold 160-2 is programmed at the controller 114 (and can be adjusted by using an external device 101 to update the value stored at the controller 114).

In response to the user activating a deflation cycle using the external device 101 (e.g., selecting a user control on the external device 101), the external device 101 may transmit a wireless signal 109 to the electronic pump assembly 106 to initiate the deflation cycle (received via the antenna 112), where the controller 114 may control the active valve(s) 118 (and, in some examples, pump 120-3) to transfer fluid from the inflatable member 104 to the fluid reservoir 102. For example, the controller 114 may control the active valve 118 to move to the open position to allow fluid to transfer from the inflatable member 104 to the fluid reservoir 102. In some examples, the controller 114 may control one or more pumps 120 to further move the fluid from the inflatable member 104 to the fluid reservoir 102 during the deflation cycle. In some examples, during the deflation cycle, fluid is transferred back until the pressure in the inflatable member 104 reaches a partial inflation threshold 162-1 (defined by the physician) or a partial inflation threshold 162-2 (defined by the patient). In some examples, the controller 114 may automatically determine to initiate a deflation cycle, which causes the controller 114 to control the active valve(s) 118 (and, in some examples, the pump 120-3) to transfer fluid back to the fluid reservoir 102.

A partial inflation pressure (e.g., partial inflation threshold 162-1, partial inflation threshold 162-2) is a pressure threshold that can more closely mimic the natural experience and/or personal comfort of the user. The partial inflation threshold 162-1 may be defined by the physician, and, in some examples, is the inflation pressure at the end of a deflation cycle. In some examples, the partial inflation pressure is in a range of 0.5 to 6.0 PSI. In some examples, the inflatable penile prosthesis 100 does not include a partial inflation threshold (162-1 or 162-2), where at the end of a deflation cycle, the pressure in the inflatable member 104 is around zero PSI. In some examples, the partial inflation threshold (162-1, 162-2) is an option that is selectable by the user (e.g., using the external device 101). In some examples, the partial inflation threshold 162-1 is programmed at the controller 114 (and can be adjusted by using an external device 101 to update the value stored at the controller 114). The partial inflation threshold 162-2 may be defined by the patient and is less than (or within the range of) the partial inflation threshold 162-1. For example, if the partial inflation threshold 162-1 is 5.0 PSI, the partial inflation threshold 162-2 can be 5.0 PSI or less than 5.0 PSI. In some examples, the partial inflation threshold 162-2 is programmed at the controller 114 (and can be adjusted by using an external device 101 to update the value stored at the controller 114).

The controller 114 may reduce the pressure 172 of the inflatable member 104 from the partial inflation threshold (e.g., 162-1, 162-2) (or a target pressure) to a lower value in response to receipt of a control signal (e.g., wireless signal 109) received via the antenna 112. For example, patients may have difficulty urinating when the inflatable member 104 is at the partial inflation threshold (e.g., 162-1, 162-2) or at the target pressure. In some examples, the user may use the external device 101 to send a wireless signal 109 to reduce the pressure 172 of the inflatable member 104 to a predetermined pressure level for urination. The controller 114 may control the active valve(s) 118 and/or the pump(s) 120 to transfer fluid from the inflatable member 104 to decrease the pressure 172 from the partial inflation threshold (e.g., 162-1, 162-2) or the target pressure to the predetermined pressure level. Then, the user may use the external device 101 to select a restore control, which transmits a wireless signal 109 to the controller 114 to restore the pressure 172 back to the partial inflation threshold (e.g., 162-1, 162-2) or the target pressure.

The controller 114 may be any type of controller configured to control operations of the pump(s) 120 and the active valve(s) 118. In some examples, the controller 114 is a microcontroller. In some examples, the controller 114 includes one or more drivers configured to drive the pump(s) 120 and the active valve(s) 118. In some examples, the driver(s) are components separate from the controller 114. The controller 114 may be communicatively coupled to the active valve(s) 118, the pump(s) 120, and the pressure sensor(s) 130. In some examples, the controller 114 is connected to the active valve(s) 118, the pump(s) 120, and the pressure sensor(s) 130 via wired data lines. The controller 114 may include a processor 113 and a memory device 115. The processor 113 may be formed in a substrate configured to execute one or more machine executable instructions or pieces of software, firmware, or a combination thereof. The processor 113 can be semiconductor-based—that is, the processors can include semiconductor material that can 2 perform digital logic. The memory device 115 may store information in a format that can be read and/or executed by the processor 113. The memory device 115 may store executable instructions that when executed by the processor 113 cause the processor 113 to perform certain operations discussed herein. The controller 114 may receive data via the pressure sensor(s) 130 and/or the external device 101 and control the active valve(s) 118 and/or the pump(s) 120 by transmitting control signals to the active valve(s) 118 and/or the pump(s) 120.

The memory device 115 may store control parameters that can be set or modified by the user and/or physician using the external device 101. In some examples, the memory device 115 may store the maximum pressure threshold 160-1, the maximum pressure threshold 160-2, the partial inflation threshold 162-1, and/or the partial inflation threshold 162-2. A user or physician may update the control parameters using the external device 101, which can be communicated to the controller 114 via the antenna 112 and then updated in the memory device 115. In some examples, the controller 114 may store usage statistics 164 in the memory device 115. The usage statistics 164 may include one or more statistics on the usage of the inflatable penile prosthetic 100. For example, the usage statistics 164 may include the number of inflations (e.g., erections), the target pressures (which pressure the inflatable member 104 was inflated to) and/or battery conditions of the battery 116. In some examples, the controller 114 may periodically transmit the usage statistics 164 to an external device 101.

The external device 101 may communicate with the electronic pump assembly 106 over a network. In some examples, the network includes a short-range wireless network such as near field communication (NFC), Bluetooth, or infrared communication. In some examples, the network may include the Internet (e.g., Wi-Fi) and/or other types of data networks, such as a local area network (LAN), a wide area network (WAN), a cellular network, satellite network, or other types of data networks.

In some examples, the electronic pump assembly 106 includes a single pump 120 such as pump 120-1. The pump 120-1 may be disposed in parallel with the active valve 118. In some examples, the electronic pump assembly 106 includes multiple pumps 120. For example, the pumps 120 may include pump 120-1 and pump 120-2. In some examples, the pump 120-1 is disposed in a fluid passageway 125 that is used to fill the inflatable member 104 (e.g., during the inflation cycle). In some examples, the pump 120-2 is disposed in a parallel fluid passageway 127 that is used to fill the inflatable member 104 (e.g., during the inflation cycle). In some examples, the pump 120-2 is disposed in parallel with the pump 120-1. The pump 120-1 may transfer fluid according to a first flow rate, and the pump 120-1 may transfer fluid according to a second flow rate. In some examples, the first flow rate is substantially the same as the second flow rate. In some examples, the first flow rate is different from the second flow rate.

In some examples, the pumps 120 may include pump 120-3 disposed in series with the active valve 118. The pump 120-3 may transfer fluid from the inflatable member 104 to the fluid reservoir 102 (e.g., during a deflation cycle). For example, during a deflation cycle, the controller 114 may activate the active valve 118 to be in the open position and may activate the pump 120-3 to transfer fluid from the inflatable member 104 to the fluid reservoir 102. In some examples, the pump 120-3 may transfer fluid according to a third flow rate. In some examples, the third flow rate is less than the first flow rate and/or the second flow rate. In some examples, the electronic pump assembly 106 may include one or more series pumps 120 and one or more parallel pumps 120. The electronic pump assembly 106 may include a fourth pump in parallel with the pump 120-2, a fifth pump in parallel with the fourth pump, and so forth. In some examples, the pumps 120 may include one or more pumps 120 in series with one or more other pumps 120. For example, one or more pumps 120 may be in series with the pump 120-1. In some examples, one or more pumps 120 may be in series with the pump 120-2. In some examples, one or more pumps 120 may be in series with the pump 120-3.

Each pump 120 is an electronically-controlled pump. Each pump 120 may be electronically-controlled by the controller 114. For example, each pump 120 may be connected to the controller 114 and may receive a signal to actuate a respective pump 120. A pump 120 may be unidirectional in which the pump 120 can transfer fluid from the fluid reservoir 102 to the inflatable member 104 (or from the inflatable member 104 to the fluid reservoir 102). In some examples, a pump 120 is bidirectional in which the pump 120 can transfer fluid from the fluid reservoir 102 to the inflatable member 104 and from the inflatable member 104 to the fluid reservoir 102. In some examples, the pumps 120 are either unidirectional or bidirectional. In some examples, the pumps 120 include a combination of one or more unidirectional pumps and one or more bidirectional pumps.

In some examples, the pump 120 is an electromagnetic pump that moves the fluid between the fluid reservoir 102 and the inflatable member 104 using electromagnetism. With respect to an electromagnetic pump, a magnetic fluid is set at angles to the direction the fluid moves in, and a current is passed through it.

In some examples, the pump 120 is a piezoelectric pump. In some examples, a piezoelectric pump may be a diaphragm micropump that uses actuation of a diaphragm to drive a fluid. In some examples, a piezoelectric pump may include one or more piezo pumps (e.g., piezo elements), which may be implemented by a substrate layer (e.g., a single substrate layer) of high-voltage piezo elements or may be implemented by multiple substrate layers (e.g., stacked substrate layers) of low-voltage piezo elements. In some examples, the pump 120 includes a plurality of micro-pumps (e.g., piezoelectrically-driven micro-pumps) disposed on one or more substrates (e.g., wafer(s)). In some examples, the micro-pumps include a silicon-based material. In some examples, the micro-pumps include a metal (e.g., steel) based material. In some examples, the pump 120 is non-mechanical (e.g., without moving parts).

In some examples, in the case of multiple pumps 120, each pump 120 may be a pump of the same type (e.g., all pumps 120 are electromagnetic pumps or all pumps 120 are piezoelectric pumps). In some examples, one or more pumps 120 are different from one or more other pumps 120. For example, pumps 120 may include different types of piezoelectric pumps or the pumps 120 may include different types of electromagnetic pumps. The pump 120-1 may be a piezoelectric pump having a first number of micro-pumps, the pump 120-2 may be a piezoelectric pump having a second number of micro-pumps, and the pump 120-3 may be a piezoelectric pump having a third number of micro-pumps (where at least two (or all) of the first, second, and third numbers are different from each other or the same as each other). The pump 120-1 may be an electromagnetic pump, the pump 120-2 may be a piezoelectric pump, and the pump 120-3 may be an electromagnetic pump or a piezoelectric pump.

A pump 120 may include one or more passive check valves. The passive check valve(s) may assist with maintaining pressure in the inflatable member 104. In some examples, a pump 120 may include a single passive check valve. In some examples, the pump 120 may include multiple passive check valves such as two passive check valves or more than two passive check valves (e.g., disposed in series with each other). The passive check valve(s) of a respective pump 120 may not be directly controlled by the controller 114, but rather controlled based on the pressure between the inflatable member 104 and the fluid reservoir 102. The passive check valve(s) may transition between an open position (in which fluid is permitted to flow through the passive check valve(s)) and a closed position (in which fluid is prevented from flowing through the passive check valve(s)). In some examples, with respect to the pump 120-1 and the pump 120-2, the passive check valve(s) are forward biased to allow a passive flow through the passive check valve(s) in the direction from the fluid reservoir 102 to the inflatable member 104 while the passive check valve(s) are in the closed position in the flow direction from the inflatable member 104 to the fluid reservoir 102. In some examples, the passive check valve(s) transitions to the closed position in response to positive pressure between the inflatable member 104 and the fluid reservoir 102. In some examples, the passive check valve(s) transition to the open position in response to negative pressure between the inflatable member 104 and the fluid reservoir 102.

In some examples, the use of two parallel pumps (e.g., pump 120-1, pump 120-2) (or more than two parallel pumps 120) may increase the amount of fluid that can be transferred to the inflatable member 104. In some examples, the pumps 120 may operate out of phase from each other in order to increase the efficiency of the electronic pump assembly 106. Two parallel pumps (e.g., pump 120-1, pump 120-2) operating at out of phase (e.g., 180 degrees of out of phase) from each other may allow the output pressure of the pump 120-1 to improve the valve closure of the pump 120-2, thereby improving the overall performance (and vice versa). The use of parallel pumps 120 operating out of phase from each other may allow the pumps 120 to operate at lower frequencies, which can reduce power (thereby extending battery life). Furthermore, a smoother flow rate may also be achieved resulting in less vibration and an improved patient experience. As indicated above, one or more pumps 120 may be in series with one or more parallel pumps 120. For example, an additional pump 120 may be in series with the pump 120-1, and/or an additional pump 120 may be in series with the pump 120-2. Serial pump operation may enable doubling of the pressure when two similar-performing pumps 120 are utilized. In some examples, two or more serially-disposed pumps 120 may be operated at the same phase.

Out of phase may refer to two or more control signals whose phase relationship with each other is such that one control signal is at its positive peak while the other control signal is at (or near) its negative peak. The pump 120-1 may operate according to a first control signal (generated by the controller 114), and the pump 120-2 may operate according to a second control signal (generated by the controller 114). The first and second control signals may control the pump 120-1 and the pump 120-2, respectively, to operate out of phase from one another. Each of the first control signal and the second control signal may define a series of activation states, e.g., a first state and a second state. For example, each of the first control signal and the second control signal may include a waveform having a series of first states (one of high states or low states) and second states (one of low states or high states). The first state may indicate that a diaphragm element moves in a first direction, and the second state may indicate that the diaphragm element moves in a second direction (opposite to the first direction). The first signal may indicate the first state during a first period of time, followed by the second state during a second period of time, followed by the first state during a third period of time, followed by the second state during a fourth period of time, and so forth. The second signal may indicate the second state during the first period of time, followed by the first state during the second period time, the second state during the third period of time, the first state during the fourth period of time, and so forth.

The active valve 118 may be an electronically-controlled valve. The active valve 118 may be electronically-controlled by the controller 114. For example, the active valve 118 may be connected to the controller 114 and may receive a signal to transition the active valve 118 between an open position in which the fluid flows through the active valve 118 and a closed position in which the fluid is prevented from flowing through the active valve 118. In some examples, the active valve 118 includes a diaphragm and a ring member (e.g., an O-ring). In some examples, in the closed position, the flow path is impeded by the interface between the diaphragm and the ring member. In some examples, in the open position, the diaphragm is related from the ring member (e.g., disposed a distance apart), which permits the fluid to flow through the active valve 118. An active valve 118 may be bidirectional. Each active valve 118 may be piezo or electromagnetic diaphragm actuated. In some examples, the active valve 118 is disposed in a fluid passageway 124 that is used to empty the inflatable member 104 (e.g., in the deflation cycle). In some examples, the active valve 118 may transition to the closed position to hold (e.g., substantially hold) the pressure in the inflatable member 104. In some examples, the active valve 118 may transition to the open position to transfer fluid back to the fluid reservoir 102, release pressure in the inflatable member 104 and/or allow a flow back to the inflatable member 104. In some examples, the active valve 118 may be used to hold (e.g., substantially hold) the partial inflation pressure.

In some examples, the electronic pump assembly 106 includes a single active valve 118. In some examples, the electronic pump assembly 106 includes multiple active valves 118. In some examples, one or more additional active valves 118 may be in series with the pump 120-1, the pump 120-2, and/or the pump 120-3. In some examples, an additional active valve 118 (e.g., a series active valve 118) may be disposed in a fluid pathway portion that is connected to the fluid reservoir 102. In some examples, an additional active valve 118 (e.g., a series active valve 118) may be disposed in a fluid pathway portion that is connected to the inflatable member 104. These additional active valves 118 may reduce leakage when at maximum inflation pressure or at partial inflation pressure.

The electronic pump assembly 106 may include one or more pressure sensors 130 configured to sense a pressure of the inflatable penile prosthesis 100. In some examples, the electronic pump assembly 106 includes a single pressure sensor 130. In some examples, the electronic pump assembly 106 may include multiple pressure sensors 130.

The pressure sensor 130 is configured to measure a pressure 172 of the inflatable member 104. The controller 114 may include a pressure controller 174 that receives pressure readings from the pressure sensor 130 according to a pressure sensing rate 176. Each pressure reading may indicate the pressure 172 of the inflatable member 104 at a time of the pressure reading.

The controller 114 (e.g., the pressure controller 174) and the pressure sensor 130 may regulate the pressure 172 of the inflatable member 104. For example, in the event of compression of the inflatable member 104, a pressure increase (or spike) may occur. However, the pressure regulation discussed herein may minimize or prevent injury to the patient or damage to the inflatable penile prosthesis 100. The pressure controller 174 may control the pressure 172 of the inflatable member 104 based on a combination of pump(s) 120 and active valve(s) 118.

For example, the pressure controller 174 may receive pressure readings from the pressure sensor 130, and, in response to the measured pressure 172 being greater than a threshold, the pressure controller 174 may activate the active valve 118 to be in the open position to transfer a portion of the fluid from the inflatable member 104 back to the fluid reservoir 102. In some examples, the threshold is the maximum pressure threshold 160-1. In some examples, the threshold is the maximum pressure threshold 160-2. In some examples, the threshold is a pressure value that is greater than the maximum pressure threshold 160-1 and/or the maximum pressure threshold 160-2. In some examples, the pressure controller 174 may cause the active valve 118 to be in the open position in response to the pressure 172 being greater than the threshold for a certain period of time. For example, if the measured pressure 172 is above the threshold for more than a predefined period of time, the pressure controller 174 switches the active valve 118 to the open position. In response to the pressure 172 being detected as corresponding to the threshold (or less than the threshold), the pressure controller 174 may switch the active valve 118 to be in the closed position.

In some examples, there may be a small amount of leakage through the pump(s) 120, the passive valve(s), and the active valve(s) 118, and this leakage may increase at greater pressure escalations during sexual activity. However, in response to the measured pressure 172 being less than the target pressure, the pressure controller 174 may activate one or more pumps 120 to transfer additional fluid to the inflatable member 104 such that the target pressure can be achieved. In some examples, the target pressure is the pressure set by the patient.

In some examples, when the inflatable member 104 is inflated to its target pressure (e.g., during sexual activity), the controller 114 may increase the pressure sensing rate 176 at which the controller 114 receives pressure readings (e.g., the pressure 172 is measured at high-speed intervals during sexual activity). For example, the controller 114 may include a pressure sensing rate controller 180 configured to control (e.g., adjust) the pressure sensing rate 176. In some examples, the pressure sensing rate controller 180 sets the pressure sensing rate 176 at a first value (e.g., a higher pressure sensing rate 176) in response to the inflation cycle being activated, the pressure 172 in the inflatable member 104 being near or at the target pressure, and/or the detection of an activity level 186 and/or activity type 190 that indicates sexual activity. The activity level 186 and/or the activity type 190 may be determined based on information obtained via an accelerometer 154, which is explained later in the disclosure. In some examples, the pressure sensing rate controller 180 may adjust the pressure sensing rate 176 to a second value (e.g., a lower pressure sensing rate 176) in response to the deflation cycle being activated, the pressure 172 in the inflatable member 104 being relatively low (e.g., at the partial inflation threshold 162-1 or 162-2, or at zero PSI). For example, when the inflatable member 104 is not inflated (or not inflated to its target pressure), the pressure sensing rate controller 180 may decrease the pressuring sensing rate 176, so that power and battery energy may be saved.

In some examples, the electronic pump assembly 106 may include additional pressure sensors 130, which can be located at various positions in the electronic pump assembly 106. For example, a pressure sensor 130 may be disposed between active valves 118. In some examples, a pressure sensor 130 may be disposed between two pumps 120 connected in series. In some examples, a pressure sensor 130 may be disposed between two pumps 120 connected in parallel. In some examples, a pressure sensor 130 may be disposed between an active valve 118 and a pump 120. In some examples, a pressure sensor 130 may be connected to the fluid reservoir 102 and measure pressure of the fluid reservoir 102. The pressure sensor(s) 130 are communicatively coupled to the controller 114 such that the controller 114 can receive signals from the pressure sensor 130. In some examples, a pressure sensor 130 is configured to sense the amount of fluid transferred to the inflatable member 104 and send one or more signals to the controller 114 that indicate the amount of fluid that has been transferred.

The electronic pump assembly 106 may include an accelerometer 154 configured to measure an acceleration 182 of a user of the inflatable penile prosthesis 100. The accelerometer 154 may measure the magnitude and direction of the acceleration 182 in one or more directions. In some examples, the accelerometer 154 is a multi-axis accelerometer that can measure the magnitude and direction in multiple directions (e.g., x-direction, y-direction, and/or z-direction). The information from the accelerometer 154 may be used to determine an activity level 186. For example, the controller 114 may include an activity level detector 184 configured to receive the accelerometer readings from the accelerometer 154 (where each accelerometer reading includes the magnitude and direction of acceleration 182 in more than one direction), and the activity level detector 184 may determine an activity level 186. In some examples, a high value for the activity level 186 (over a period of time) may indicate that the user is exercising. In some examples, a low value for the activity level 186 (over a period of time) may indicate that the user is resting or sleeping.

In some examples, the controller 114 includes an activity type detector 184 configured to determine an activity type 190 based on the activity level 186 from the activity level detector 184. In some examples, the activity type 190 may be a classification on what activity the user is currently engaging in such as exercise, rest, sleep, transport, etc. In some examples, the activity level detector 184 and the activity type detector 188 are combined into a single module that receives the acceleration readings and determines the activity level 186 and/or activity type 190. In some examples, the activity level detector 184 and/or the activity type detector 188 is configured to identify a sleep pattern of a user of the inflatable penile prosthesis based on the measured acceleration 182 (and, some examples, in combination with one or more other signals such as time of day). While the user is asleep, the controller 114 may cause the inflatable member 104 to inflate (e.g., partially inflate or inflate to a target pressure).

For example, the controller 114 may include an inflation controller 194 configured to control nocturnal and/or random erections. Nocturnal penile tumescence is a spontaneous erection of the penis during sleep or waking up and may be a contribution to penis health. Nocturnal erections may be implemented by the inflation controller 194 in order to give the patient a more realistic experience. In some examples, the inflation controller 194 may cause the inflatable member 104 to inflate at a set (or random) time(s) during a period of time during the night (e.g., 3 am to 6 am). In some examples, the inflation controller 194 may cause the inflatable member 104 to inflate to a random pressure threshold, which may be stored at the inflation controller 194 (and can be changed by the user using the external device 101). In some examples, the inflation controller 194 may receive the activity type 190 from the activity type detector 188 to confirm that the user is sleeping, and the inflation controller 194 may control the pump(s) 120 and the active valve(s) 118 to inflate the inflatable member 104. In some examples, the inflation controller 194 may control the pump(s) 120 (e.g., pump 120-1 and pump 120-2) to operate at a lower frequency and/or lower pumping rate in order to not wake the patient (e.g., at a frequency and/or pumping rate that is lower than a rate used during a patient-initiated inflation cycle).

In some examples, the inflation controller 194 may cause the inflatable member 104 to inflate at a random time during the daytime (e.g., random erection). In some examples, the inflation controller 194 may receive the activity type 190 from the activity type detector 188 (or the activity level 186 from the activity level detector 184) to ensure that the user is not engaging in activity such as exercise, and then proceed to activate the pump(s) 120 (e.g., pump 120, pump 120-2) to inflate the inflatable member 104. In some examples, the inflation controller 194 may inflate the inflatable member 104 to the random pressure threshold. In some examples, the times of the nocturnal and/or random erections may be controlled by the patient, e.g., the patient may select the appropriate times for the nocturnal and/or random erections to occur.

In some examples, the controller 114 may include a testing period controller 192 configured to control inflation and deflation cycles after the inflatable penile prosthesis 100 has been implanted into the body of the patient. For example, after the inflatable penile prosthesis 100 has been implemented into the body, a healing period is required. After the healing period, a patient may be instructed to start cycling their device through inflation and deflation. However, according to the embodiments discussed herein, the testing period controller 192 may implement a smart process that automatically implements the inflation/deflation cycles at controlled increments. For example, the testing period controller 192 may iteratively initiate an inflation cycle and a deflation cycle during a testing duration (e.g., over day(s), week(s), month(s)). In some examples, the testing duration includes a series of sub-durations (e.g., four one-week periods) in which inflation and deflation settings are adjusted. For example, at each subsequent sub-duration, the pressure 172 in the inflatable member 104 is adjusted (e.g., increased).

For example, at a first sub-duration, the testing period controller 192 may inflate the inflatable member 104 to a first pressure, hold the inflatable member 104 at the first pressure, and then deflate the inflatable member 104. In some examples, when activated, the testing period controller 192 controls the inflation and deflation and the timing of when to deflate. In some examples, the testing period controller 192 may set the target pressure to the first pressure, and the user is allowed to control the inflation, deflation, and the timing but the maximum pressure is the first pressure. At a second sub-duration (e.g., the next week), the testing period controller 192 may inflate the inflatable member 104 to a second pressure, hold the inflatable member 104 at the second pressure, and then deflate the inflatable member 104. In some examples, the second pressure is higher than the first pressure. In some examples, when activated, the testing period controller 192 controls the inflation and deflation and the timing of when to deflate. In some examples, the testing period controller 192 may set the target pressure to the second pressure, and the user is allowed to control the inflation, deflation, and the timing but the maximum pressure is the second pressure. At a third sub-duration, the testing period controller 192 may inflate the inflatable member 104 to a third pressure, hold the inflatable member 104 at the third pressure, and then deflate the inflatable member 104. In some examples, the third pressure is higher than the second pressure. In some examples, when activated, the testing period controller 192 controls the inflation and deflation and the timing of when to deflate. In some examples, the testing period controller 192 may set the target pressure to the third pressure, and the user is allowed to control the inflation, deflation, and the timing but the maximum pressure is the second pressure.

During each sub-duration, the testing period controller 192 may execute a plurality of iterations. Also, the testing period controller 192 may control the timing of when to initiate each iteration. For example, after a first iteration, the testing period controller 192 may wait a first period of time before initiating the second iteration, and then wait a second period of time before initiating the third iteration, and so forth. The time between the iterations may be the same or may be different (e.g., the time between iterations may decrease as the number of iterations increase, or the time between iterations may increase as the number of iterations increase). Also, at each iteration, the testing period controller 192 may control the amount of time the inflatable member 104 is held at a respective pressure (e.g., decreasing the time between an inflation cycle and a deflation cycle as the number of iterations increase, or increasing the time between an inflation cycle and deflation cycle as the number of iterations increase).

The electronic pump assembly 106 may include a temperature sensor 152 configured to measure a temperature 191 of the fluid in the electronic pump assembly 106. In some examples, the temperature sensor 152 is included as part of the pressure sensor 130. In some examples, the temperature sensor 152 is a sensor that is separate from the pressure sensor 130. The temperature sensor 152 may be connected to a portion of a fluid passageway in the electronic pump assembly 106. In response to the measured temperature 191 being greater than a threshold, the controller 114 may transmit, over a network, a notification message 198 via the antenna 112 to one or more external devices 101. For example, the controller 114 may include a notification generator 196 configured to generate a notification message 198 to be transmitted to one or more external devices 101. The notification message 198 may indicate a warning about the patient's health, the use of the inflatable penile prosthesis 100, and/or the high level of the temperature 191. The notification generator 196 may generate a notification message 198 when the measured temperature 191 is greater than a threshold. The notification message 198 may be transmitted to a patient's device and/or a device associated with the physician. The temperature 191 having a value over a threshold level may be an indicator of an infection around the implanted device or a possible malfunction with the device.

The controller 114 may detect a performance issue associated with the inflatable penile prosthesis 100 based on pressure readings from the pressure sensor 130. For example, leaks (e.g., a leakage event) may be detected based on the pressure readings from the pressure sensor 130. In addition, pump and active valve performance may be inferred from the pressure readings from the pressure sensor 130. For example, the pressure readings may indicate that the pump(s) 120 and/or the active valve(s) 118 are not operating properly. In some examples, the controller 114 may detect a performance issue if a pump takes longer than a threshold amount of time to reach a certain pressure. For example, the controller 114 may use the pressure readings from the pressure sensor 130 along with a time scale to detect a performance issue with one or more of the pumps. In response to the detection of a performance issue based on the pressure readings, the notification generator 196 may transmit, over a network, a notification message 198 to one or more external devices 101, where the notification message 198 may indicate that the penile prosthesis 100 has one or more performance issues.

The electronic pump assembly 106 may include one or more sensors 158 configured to monitor a performance of the battery 116, the controller 114, the accelerometer 154, a heart rate monitor 156, and/or other electronics on the electronic pump assembly 106. Based on the data received via one or more sensors 158, the controller 114 may determine whether one or more performance metrics 193 associated with the battery 116, the controller 114, the accelerometer 154, the heart rate monitor 156, and/or other electronics do not achieve a target threshold. If a performance metric 193 does not achieve a target threshold, the notification generator 196 may generate and transmit a notification message 198, over a network, to one or more external devices 101, where the notification message 198 may indicate a performance issue with one of the electronics on the electronic pump assembly 106.

The electronic pump assembly 106 may include a heart rate monitor 156 configured to monitor a heart rate of a user of the inflatable penile prosthesis 100. In some examples, the controller 114 is configured to record heart rate data 166 and store the heart rate data on the memory device 115. In some examples, the heart rate data 166 may include information about the heart rate when the inflatable member 104 is inflated and/or during sexual activity. In some examples, the heart rate data 166 may correlate the pressure 172 from the pressure readings with the heart rate of the user. In some examples, the heart rate data 166 may associate pressure fluctuations (received from the pressure sensor 130 with the heart rate. In some examples, the heart rate data 166 may associate acceleration 182 from the acceleration readings from the accelerometer 154 with the heart rate. The controller 114 may periodically transmit the heart rate data 166 to one or more external devices 101.

The controller 114 may partially inflate the inflatable member 104 such that the pressure 172 of the inflatable member 104 does not exceed a threshold (e.g., zero PSI). For example, the controller 114 may include a partial inflation controller 195 that controls the pump(s) 120 and the active valve(s) to inflate the inflatable member 104 such that the inflatable member 104 does not exceed a threshold (e.g., zero PSI). The inflatable member 104 may be empty or full and yet both be at zero PSI. In some examples, the partial inflation controller 195 may actuate the active valve 118 to the closed position and cause the pump(s) 120 to operate to fill the inflatable member 104 with fluid but still keeping the pressure 172 at zero PSI (or substantially around zero PSI (e.g., one or two PSI)). The inflatable penile prosthesis 100 may maintain pressure with partial inflation at equilibrium in static conditions and no leakage will occur as there is no pressure differential.

In some examples, the fill pumps 120 (e.g., pump 120-1, pump 120-2) are forward biased. In the event of a positive pressure differential between the fluid reservoir 102 and the inflatable member 104, some fluid (e.g., leakage fluid) may move through the passive valve(s) of a respective pump 120. This may occur, for example, during certain gym workouts and other exercises where abdominal pressure may be applied to the fluid reservoir. The forward pressure through the forward biased pumps may cause an accumulation of pressure into the inflatable member 104, which may cause an unintended partial inflation for the patient.

The controller 114 may include a pressure release controller 168 configured to coordinate the activation of the emptying pump (e.g., pump 120-3) and/or the opening of the active valve 118 with pressure pulsations 170. The pulsations 170 may be measured by the pressure sensor 130. A pulsation 170 may be a short increase of pressure 172. Just after a pulsation 170, the pressure release controller 168 may time the opening of the active valve 118 to release pressure 172 from the inflatable member 104. This is followed by a closure of the active valve 118 before the next pulsation 170. For example, the pressure release controller 168 may obtain pressure readings from the pressure sensor 130 over time according to a pressure sensor rate 176. The pressure release controller 168 may detect a first pressure pulsation 170-1 at a first time and a second pressure pulsation 170-2 at a second time. The pressure release controller 168 may cause the active valve 118 to be in an open position (and, in some examples, activate the pump 120-3) after the first time. In some examples, the pressure release controller 168 actuates the pump 120-3 at a frequency lower than a frequency used to drive the pump 120-3 during a normal deflation cycle. The pressure release controller 168 may cause the active valve 118 to be in a closed position (and, in some examples, deactivate the pump 120-3) before the second time.

The pressure release controller 168 may empty fluid from the inflatable member 104 after a period of high activity by causing the active valve 118 to be in the open position, and, in some examples, causing the pump 120-3 to operate. For example, as indicated above, the forward pressure through the forward biased pumps may cause an accumulation of pressure into the inflatable member 104, which may cause an unintended partial inflation for the patient. However, the pressure release controller 168 may receive the pressure readings from the pressure sensor 130 and the activity level 186 and/or the activity type 190. For example, during a first period of time, the activity level 186 may be relatively high indicating an activity type 164-1 of exercise. During a second period of time, the activity level 186 may be relatively low indicating an activity type 164-2 of resting. In some examples, at the end of the first period of time (or the beginning of the second period of time), the pressure release controller 168 may open the active valve 118 and actuate the pump 120-3. In some examples, the pressure release controller 168 may drive the pump 120-3 at a frequency lower than a frequency used during a deflation cycle.

The electronic pump assembly 106 may include a hermetic enclosure 108 that encloses the components of the electronic pump assembly 106. A hermetic enclosure 108 may be an air-tight (or substantially air-tight) container. The hermetic enclosure 108 may include one or more metal-based materials. In some examples, the hermetic enclosure 108 is a Titanium container. In some examples, the only material in contact with the patient is Titanium. In some examples, the hermetic enclosure 108 defines a feedthrough 140 (e.g., a hermetic feedthrough, an electrical feedthrough, a feedthrough connector, etc.) to receive/transmit wireless signals from/to the external device 101. In some examples, the feedthrough 140 includes a metal-based material and an insulator-based material (e.g., ceramic).

The electronic pump assembly 106 may include a hermetic fluid chamber 110 disposed inside of the hermetic enclosure 108. The hermetic fluid chamber 110 may be a separate air-tight (or substantially air-tight) container that is within the hermetic enclosure 108. The hermetic fluid chamber 110 may include one or more metal-based materials. In some examples, the hermetic fluid chamber 110 is a Titanium container. In some examples, the hermetic fluid chamber 110 includes one or more non-metal-based materials (e.g., ceramic). In some examples, a portion of the hermetic fluid chamber 110 is a metal-based material (e.g., Titanium) and a portion of the hermetic fluid chamber 110 is a non-metal-based material (e.g., ceramic). The hermetic fluid chamber 110 may isolate the fluid from the electronics (e.g., the controller 114, the accelerometer 154, the heart rate monitor 156, one or more sensors 158, the battery 116, etc.). In other words, the electronics section may be isolated (e.g., completely isolated) from the fluid via the hermetic fluid chamber 110. The hermetic fluid chamber 110 may be fluidly connected to the fluid reservoir 102 and the inflatable member 104. The hermetic fluid chamber 110 may include the active valve(s) 118, the pump(s) 120, the pressure sensor(s) 130, and the temperature sensor 152. In some examples, the hermetic fluid chamber 110 defines a feedthrough 138 (e.g., a hermetic feedthrough, an electrical feedthrough, a feedthrough connector, etc.) to the controller 114 to receive/transmit signals from/to the controller 114. In some examples, the hermetic fluid chamber 110 disposed within the hermetic enclosure 108 creates a double hermetic system. In some examples, the electronic pump assembly 106 includes only one hermetic enclosure (e.g., the hermetic enclosure 108).

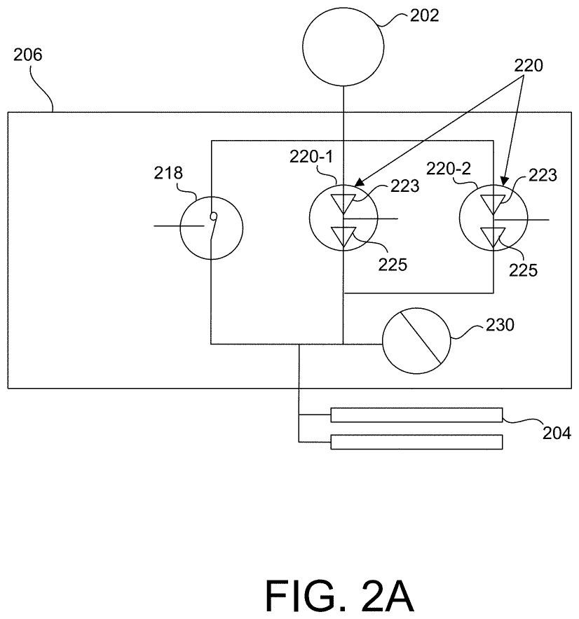

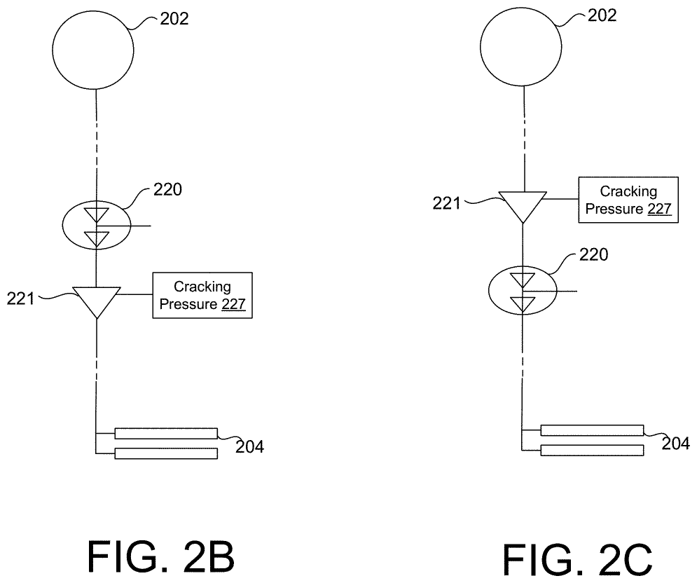

FIG. 2A illustrates an electronic pump assembly 206 according to an aspect. FIG. 2B illustrates a check valve 221 disposed in series with one of the pumps 220 according to an aspect. FIG. 2C illustrates a check valve 221 disposed in series with one of the pumps 220 according to another aspect. The electronic pump assembly 206 may be an example of the electronic pump assembly 106 of FIGS. 1A and 1B and may include any of the details discussed with reference to those figures.

The electronic pump assembly 206 includes an active valve 218, a pump 220-1, a pump 220-2 and a pressure sensor 230. The pump 220-1 may include an inlet and an outlet. The inlet of the pump 220-1 may be fluidly connected to a fluid reservoir 202, and the outlet of the pump 220-1 may be fluidly connected to an inflatable member 204. The pump 220-2 may include an inlet and an outlet. The inlet of the pump 220-2 may be fluidly connected to the fluid reservoir 202, and the outlet of the pump 220-2 may be fluidly connected to the inflatable member 204. The active valve 218 may include an inlet and an outlet. The inlet of the active valve 218 may be fluidly connected to the inflatable member 204 and the outlet of the active valve 218 may be fluidly connected to the fluid reservoir 202.

The pump 220-1 and the pump 220-2 are electronically-controlled pumps. The pump 220-1 and the pump 220-2 may be electronically-controlled by a controller (e.g., the controller 114 of FIGS. 1A and 1B). In some examples, the pump 220-1 and the pump 220-2 are unidirectional in which the pump 220-1 and the pump 220-2 can transfer fluid from the fluid reservoir 202 to the inflatable member 204. However, in some examples, the pump 220-1 and the pump 220-2 are bidirectional. In some examples, the pump 220-1 or the pump 220-2 is an electromagnetic pump or a piezoelectric pump.

The pump 220-1 or the pump 220-2 may include a passive check valve 223 and a passive check valve 225. The passive check valve 223 and the passive check valve 225 may assist with maintaining pressure in the inflatable member 204. The pump 220-1 may be disposed in parallel with the active valve 218. The pump 220-2 may be disposed in parallel with the pump 220-1. In some examples, the use of two parallel pumps (e.g., pump 220-1, pump 220-2) may increase the amount of fluid that can be transferred to the inflatable member 204. In some examples, the pump 220-1 and the pump 220-1 may operate out of phase from each other in order to increase the efficiency of the electronic pump assembly 206. In some examples, two parallel pumps (e.g., pump 220-1, pump 220-2) operating out of phase (e.g., 180 degrees of out of phase) from each other may allow the output pressure of the pump 220-1 to improve the valve closure of the pump 220-2, thereby improving the overall performance (and vice versa). In some examples, the use of parallel pumps (e.g., pump 220-1, pump 220-2) operating out of phase from each other may allow the pump 220-1 and the pump 220-2 to operate at lower frequencies, which can reduce power (thereby extending battery life). Furthermore, a smoother flow rate may also be achieved resulting in less vibration and an improved patient experience.

The active valve 218 may be an electronically-controlled valve. The active valve 218 may be electronically-controlled by a controller (e.g., the controller 114 of FIGS. 1A and 1B). For example, the active valve 218 may receive a signal to transition the active valve 218 between an open position in which the fluid flows through the active valve 218 and a closed position in which the fluid is prevented from flowing through the active valve 218. In some examples, the active valve 218 may transition to the closed position to hold (e.g., substantially hold) the pressure in the inflatable member 204. In some examples, the active valve 218 may transition to the open position to transfer fluid back to the fluid reservoir 202, release pressure in the inflatable member 204 and/or allow a flow back to the inflatable member 204. In some examples, the active valve 218 may be used to hold (e.g., substantially hold) the partial inflation pressure.

The pressure sensor 230 is configured to measure the pressure of the inflatable member 204. The pressure sensor 230 may be coupled to a portion of the fluid passageway connected to the inflatable member 204. In some examples, the pressure sensor 230 may be coupled to a portion of the fluid passageway between the active valve 218 and the inflatable member 204. In some examples, the pressure sensor 230 may be coupled to a portion of the fluid passageway between the pump 220-1 and the inflatable member 204. In some examples, the pressure sensor 230 may be coupled to a portion of the fluid passageway between the pump 220-2 and the inflatable member 204. The pressure sensor 230 is communicatively coupled to a controller (e.g., the controller 114 of FIGS. 1A and 1B) such that the controller can receive signals from the pressure sensor 230.

In some examples, as shown in FIGS. 2C and 2C, the electronic pump assembly 206 may include a check valve 221 disposed in series with a pump 220 (where the pump 220 can be pump 220-1 or pump 220-2 or both). In some examples, as shown in FIG. 2B, the check valve 221 is disposed between the pump 220 and the inflatable member 204. In some examples, as shown in FIG. 2C, the check valve 221 is disposed between the pump 220 and the fluid reservoir 202. The check valve 221 may define a cracking pressure 227. When the pressure differential between the inlet of the check valve 221 and the outlet of the check valve 221 is greater than the cracking pressure 227, the check valve 221 is configured to open to permit the passage of fluid. When the pressure differential between the inlet of the check valve 221 and the outlet of the check valve 221 is less than the cracking pressure 227, the check valve 221 is configured to close, thereby blocking the transfer of fluid. In some examples, the cracking pressure 227 is in a range of 1.0 PSI to 5.0 PSI. In some examples, the cracking pressure 227 is in a range of 2.0 PSI to 4.0 PSI. In some examples, the cracking pressure 227 is 3.0 PSI. A check valve 221 placed in series with a pump 220 may reduce leakage or inadvertent flow from the fluid reservoir 202 to the inflatable member 204 during activity which places abdominal pressure on the fluid reservoir 202. In some examples, the check valve 221 is closed during normal non-erection periods and ensures that inadvertent partial erections are minimized.

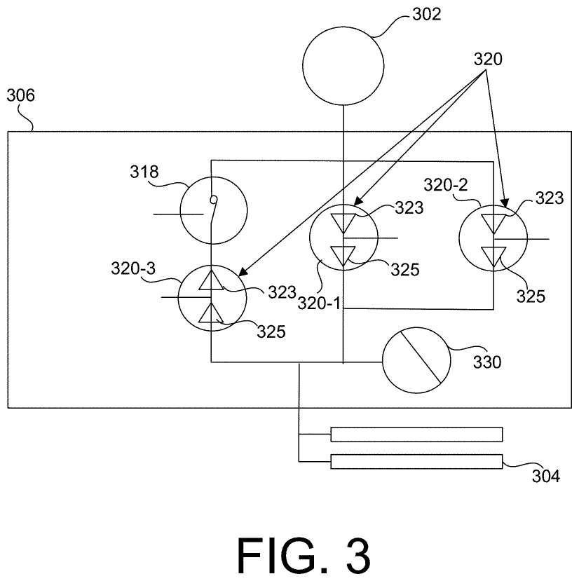

FIG. 3 illustrates an electronic pump assembly 306 according to an aspect. The electronic pump assembly 306 may be an example of the electronic pump assembly 106 of FIGS. 1A and 1B and/or the electronic pump assembly 206 of FIGS. 2A through 2C and may include any of the details discussed with reference to those figures. The electronic pump assembly 306 may be the same (similar) to the electronic pump assembly 206 of FIGS. 2A through 2C except that an emptying pump (e.g., pump 320-3) is included on the electronic pump assembly 306.

The electronic pump assembly 306 includes an active valve 318, a pump 320-1, a pump 320-2, a pump 320-3, and a pressure sensor 230. The pump 320-1 may include an inlet and an outlet. The inlet of the pump 320-1 may be fluidly connected to a fluid reservoir 302, and the outlet of the pump 320-1 may be fluidly connected to an inflatable member 304. The pump 320-2 may include an inlet and an outlet. The inlet of the pump 320-2 may be fluidly connected to the fluid reservoir 302, and the outlet of the pump 320-2 may be fluidly connected to the inflatable member 304. The pump 320-3 may include an inlet and an outlet. The inlet of the pump 320-3 may be fluidly connected to the inflatable member, and the outlet of the pump 320-3 may be fluidly connected to the active valve 318 (and the fluid reservoir 302). The active valve 318 may include an inlet and an outlet. The inlet of the active valve 318 may be fluidly connected to the outlet of the pump 320-2 (and the inflatable member 304) and the outlet of the active valve 318 may be fluidly connected to the fluid reservoir 302.

The pump 320-1, the pump 320-2, and the pump 320-3 are electronically-controlled pumps. The pump 320-1, the pump 320-2, and the pump 320-3 may be electronically-controlled by a controller (e.g., the controller 114 of FIGS. 1A and 1B). In some examples, the pump 320-1 and the pump 320-2 are unidirectional in which the pump 320-1 and the pump 320-2 can transfer fluid from the fluid reservoir 302 to the inflatable member 304. In some examples, the pump 320-3 is unidirectional in which the pump 320-3 can transfer fluid from the inflatable member 304 to the fluid reservoir 302. However, in some examples, the pump 320-1, the pump 320-2, and the pump 320-3 are bidirectional. In some examples, the pump 320-1, the pump 320-2, and the pump 320-3 may be electromagnetic pumps or piezoelectric pumps.

The pump 320-1, the pump 320-2, or the pump 320-3 may include a passive check valve 323 and a passive check valve 325. The passive check valve 323 and the passive check valve 325 may assist with maintaining pressure in the inflatable member 304. The pump 320-1 may be disposed in parallel with the active valve 318. The pump 320-2 may be disposed in parallel with the pump 320-1. The pump 320-3 may be disposed in series with the active valve 318. The pump 320-3 may be disposed in parallel with the pump 320-1 or the pump 320-2.

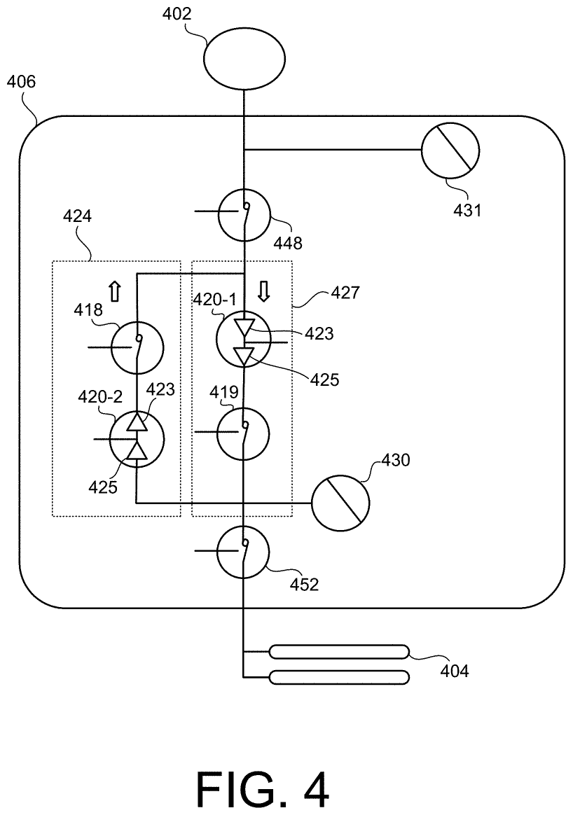

FIG. 4 illustrates an example of a portion of an electronic pump assembly 406 according to an aspect. The electronic pump assembly 406 may be an example of the electronic pump assembly 106 of FIGS. 1A and 1B, the electronic pump assembly 206 of FIGS. 2A through 2C, and/or the electronic pump assembly 306 of FIG. 3 and may include any of the details discussed with reference to those figures.

The electronic pump assembly 406 is configured to transfer fluid between the fluid reservoir 402 and the inflatable member 404. The electronic pump assembly 406 may automatically transfer fluid between the fluid reservoir 402 and the inflatable member 404 without the user manually operating a pump (e.g., squeezing and releasing a pump bulb).

The electronic pump assembly 406 includes a pump 420-1 disposed within a fluid passageway 424 (e.g., a fill passageway), and an active valve 418 disposed within a fluid passageway 427 (e.g., an empty passageway). The pump 420-1 may be an electromagnetic pump or a Piezoelectric pump. The pump 420-1 may include a passive check valve 423 and a passive check valve 425. The fluid passageway 427 may be a fluid branch that is separate (and parallel) to the fluid passageway 424. The fluid passageway 427 is the passageway that transfers fluid from the fluid reservoir 402 to the inflatable member 404. The fluid passageway 424 is the passageway that transfers fluid from the inflatable member 404 to the fluid reservoir 402. The pump 420-1 is disposed in parallel with the active valve 418.

In some examples, the electronic pump assembly 406 may include an active valve 419 in series with the pump 420-1 (e.g., the pump 420-1 and the active valve 419 are disposed within the fluid passageway 427). In some examples, the electronic pump assembly 406 may include a pump 420-2 in series with the active valve 418 (e.g., the pump 420-2 and the active valve 418 are disposed in the fluid passageway 424). The pump 420-2 may be an electromagnetic pump or a Piezoelectric pump. The pump 420-2 may include a passive check valve 423 and a passive check valve 425. In some examples, the electronic pump assembly 406 includes an active valve 448 that is fluidly connected to the fluid reservoir 402. The active valve 448 may be in series with either the active valve 418 (and the pump 420-2) or the pump 420-1 (and the active valve 419). In some examples, the electronic pump assembly 406 includes an active valve 452 that is fluidly connected to the inflatable member 404. The active valve 452 may be in series with either the active valve 419 (and the pump 420-1) or the pump 420-2 (and the active valve 418).

The active valve 448, the pump 420-1, the active valve 418, the active valve 452, the active valve 418, and the pump 420-2 may be electronically controlled by a controller and/or driver (e.g., the controller 114 of FIGS. 1A and 1B). The pump 420-1 and the pump 420-2 may be unidirectional or bidirectional. With respect to the fluid passageway 427, in some examples, the pump 420-1 and the active valve 419 may swap positions (e.g., where the active valve 419 is in series between the active valve 448 and the pump 420-1). With respect to the fluid passageway 424, in some examples, the active valve 418 and the pump 420-2 may swap positions (e.g., where the pump 420-1 is in series with and between the active valve 418 and the active valve 448).

In some examples, one or more additional active valves and/or one or more additional pumps are disposed in series within the fluid passageway 427. In some examples, one or more additional active valves and/or one or more additional pumps are disposed in series within the fluid passageway 424. In some examples, the electronic pump assembly 406 may include one or more additional (and parallel) fluid passageways, where each additional (and parallel) fluid passageway may include one or more active valves and one or more pumps.

In some examples, the electronic pump assembly 406 may include a pressure sensor 430 and a pressure sensor 431. The pressure sensor 430 and the pressure sensor 431 are connected to a controller (e.g., the controller 114 of FIGS. 1A and 1B), where the controller receives the measured pressure from the pressure sensor 430 and the pressure sensor 431.

The pressure sensor 430 is configured to measure the pressure in the inflatable member 404. The controller may receive the measured pressure from the pressure sensor 430 and automatically control the active valves and/or the pump to regulate the pressure. In some examples, the pressure sensor 431 is configured to measure the pressure in the fluid reservoir 402. In some examples, the pressure sensor 431 may detect intra-abdominal pressure (which can increase during activities such as exercise, and the controller can control the active valves and pump to minimize or prevent accidental inflations. In some examples, the electronic pump assembly 406 may include one or more pressure sensors at other locations within the electronic pump assembly 406. For example, a pressure sensor may be disposed between the active valve 448 and the pump 420-1. In some examples, a pressure sensor may be disposed between the pump 420-1 and the active valve 419. In some examples, a pressure sensor may be disposed between the active valve 448 and the active valve 418. In some examples, a pressure sensor may be disposed between the active valve 418 and the pump 420-2.

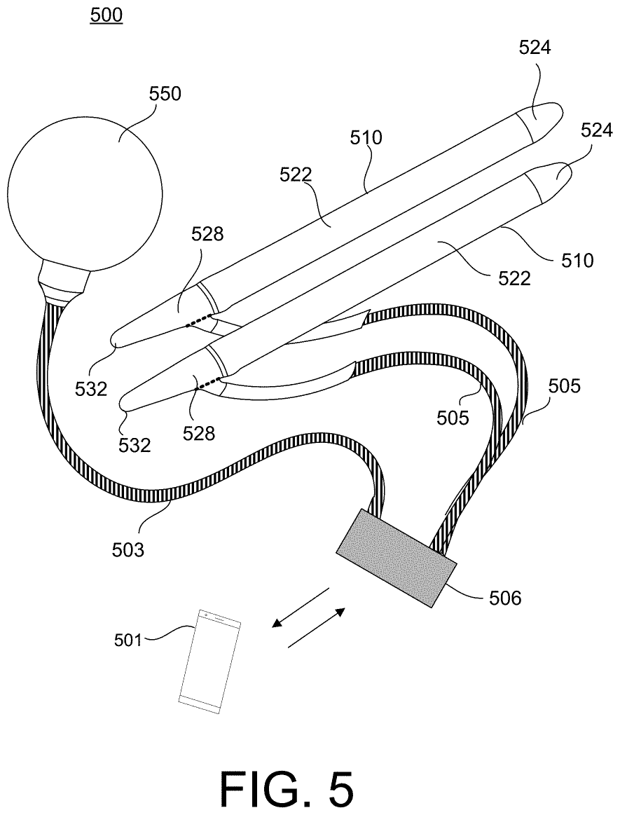

FIG. 5 schematically illustrates an inflatable penile prosthesis 500 having an electronic pump assembly 506 according to an aspect. The electronic pump assembly 506 may include any of the features of the electronic pump assembly (e.g., 106, 206, 306, 406) and the inflatable penile prostheses (e.g., 100, 200) discussed herein. The inflatable penile prosthesis 500 may include a pair of inflatable cylinders 510, and the inflatable cylinders 510 are configured to be implanted in a penis. For example, one of the inflatable cylinders 510 may be disposed on one side of the penis, and the other inflatable cylinder 510 may be disposed on the other side of the penis. Each inflatable cylinder 510 may include a first end portion 524, a cavity or inflation chamber 522, and a second end portion 528 having a rear tip 532.

At least a portion of the electronic pump assembly 506 may be implanted in the patient's body. A pair of conduit connectors 505 may attach the electronic pump assembly 506 to the inflatable cylinders 510 such that the electronic pump assembly 506 is in fluid communication with the inflatable cylinders 510. Also, the electronic pump assembly 506 may be in fluid communication with a fluid reservoir 550 via a conduit connector 503. The fluid reservoir 550 may be implanted into the user's abdomen. The inflation chamber 522 of the inflatable cylinder 510 may be disposed within the penis. The first end portion 524 of the inflatable cylinder 510 may be at least partially disposed within the crown portion of the penis. The second end portion 528 may be implanted into the patient's pubic region PR with the rear tip 532 proximate to the pubic bone PB.

In order to implant the inflatable cylinders 510, the surgeon first prepares the patient. The surgeon often makes an incision in the penoscrotal region, e.g., where the base of the penis meets with the top of the scrotum. From the penoscrotal incision, the surgeon may dilate the patient's corpus cavernosum to prepare the patient to receive the inflatable cylinders 510. The corpus cavernosum is one of two parallel columns of erectile tissue forming the dorsal part of the body of the penis, e.g., two slender columns that extend substantially the length of the penis. The surgeon will also dilate two regions of the pubic area to prepare the patient to receive the second end portion 528. The surgeon may measure the length of the corpora cavernosum from the incision and the dilated region of the pubic area to determine an appropriate size of the inflatable cylinders 510 to implant.

After the patient is prepared, the penile prosthesis 500 is implanted into the patient. The tip of the first end portion 524 of each inflatable cylinder 510 may be attached to a suture. The other end of the suture may be attached to a needle member (e.g., Keith needle). The needle member is inserted into the incision and into the dilated corpus cavernosum. The needle member is then forced through the crown of the penis. The surgeon tugs on the suture to pull the inflatable cylinder 510 into the corpus cavernosum. This is done for each inflatable cylinder 510 of the pair. Once the inflation chamber 522 is in place, the surgeon may remove the suture from the tip. The surgeon then inserts the second end portion 528. The surgeon inserts the rear end of the inflatable cylinder 510 into the incision and forces the second end portion 528 toward the pubic bone PB until each inflatable cylinder 510 is in place.

A user may use an external device 501 to control the inflatable penile prosthesis 500. In some examples, the user may use the external device 501 to inflate or deflate the inflatable cylinders 510. For example, in response to the user activating an inflation cycle using the external device 501, the external device 501 may transmit a wireless signal to the electronic pump assembly 506 to initiate the inflation cycle to transfer fluid from the fluid reservoir 550 to the inflatable cylinders 510. In some examples, in response to the user activating a deflation cycle using the external device 501, the external device 501 may transmit a wireless signal to the electronic pump assembly 506 to initiate the deflation cycle to transfer fluid from the inflatable cylinders 510 to the fluid reservoir 550. In some examples, during the deflation cycle, fluid is transferred back until the pressure in the inflatable cylinders 510 reaches a partial inflation pressure.

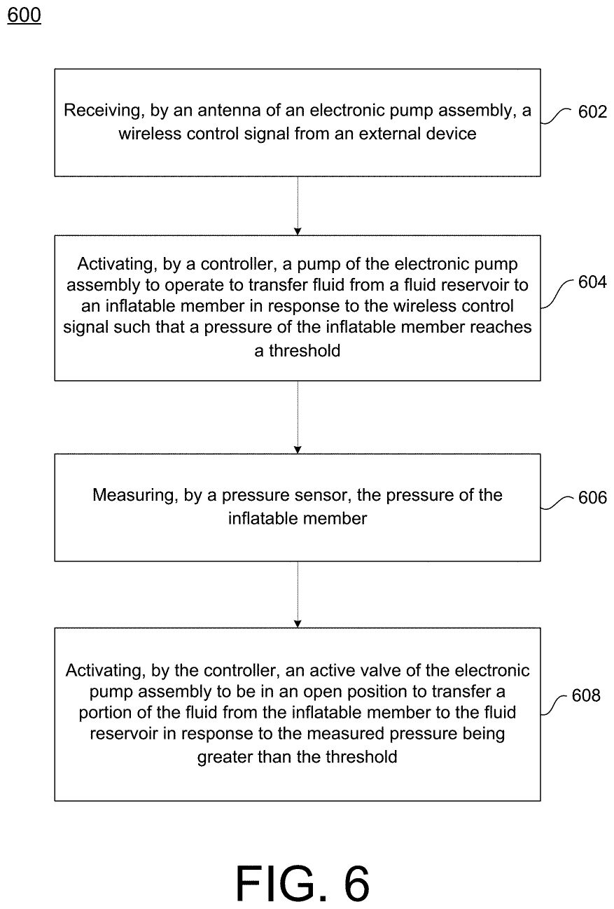

FIG. 6 illustrates a flow chart 600 depicting example operations of a method of operating an electronic pump assembly of an inflatable penile prosthesis. The example operations of the flow chart 600 may be performed by any of the inflatable penile prostheses and/or the electronic pump assemblies (e.g., 106, 206, 306, 406, 506) discussed herein.

Operation 602 includes receiving, by an antenna of an electronic pump assembly, a wireless control signal from an external device. Operation 604 includes activating, by a controller, a pump of the electronic pump assembly to operate to transfer fluid from a fluid reservoir to an inflatable member in response to the wireless control signal such that a pressure of the inflatable member reaches a threshold. Operation 606 includes measuring, by a pressure sensor, the pressure of the inflatable member. Operation 608 includes activating, by the controller, an active valve of the electronic pump assembly to be in an open position to transfer a portion of the fluid from the inflatable member to the fluid reservoir in response to the measured pressure being greater than the threshold. In some examples, the operations include detecting, by the controller, that the measured pressure corresponds to the threshold. In some examples, the operations include activating, by the controller, the active valve to be in a closed position in response to the measured pressure being detected as corresponding to the threshold.

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human.

While certain features of the described implementations have been illustrated as described herein, many modifications, substitutions, changes, and equivalents will now occur to those skilled in the art. It is, therefore, to be understood that the appended claims are intended to cover all such modifications and changes as fall within the scope of the embodiments.

What is claimed is:

1. An inflatable penile prosthesis comprising:
a fluid reservoir configured to hold fluid;
an inflatable member; and
an electronic pump assembly configured to transfer the fluid between the fluid reservoir and the inflatable member, the electronic pump assembly including:
a pump;
an active valve;
a pressure sensor configured to measure a pressure of the inflatable member; and
a controller configured to control at least one of the pump or the active valve based on the measured pressure.

2. The inflatable penile prosthesis of claim 1, wherein the controller is configured to cause the active valve to be in an open position to transfer a portion of the fluid from the inflatable member to the fluid reservoir in response to the measured pressure being greater than a threshold.

3. The inflatable penile prosthesis of claim 1, wherein the controller is configured to cause the pump to operate to transfer a portion of the fluid from the fluid reservoir to the inflatable member in response to the measured pressure being less than a threshold.

4. The inflatable penile prosthesis of claim 1, wherein the fluid reservoir includes a pressure-regulating flexible member configured to cause the inflatable member to be partially inflated without operating the pump.

5. The inflatable penile prosthesis of claim 1, wherein the electronic pump assembly includes an accelerometer configured to measure an acceleration of a user of the inflatable penile prosthesis.

6. The inflatable penile prosthesis of claim 5, wherein the controller is configured to identify a sleep pattern of a user of the inflatable penile prosthesis based on the measured acceleration, the controller configured to cause the inflatable member to at least partially inflate when the user is asleep.

7. The inflatable penile prosthesis of claim 1, wherein the electronic pump assembly includes a heart rate sensor configured to monitor a heart rate of a user of the inflatable penile prosthesis.

8. The inflatable penile prosthesis of claim 1, wherein the electronic pump assembly includes a temperature sensor configured to measure a temperature of the fluid.

9. The inflatable penile prosthesis of claim 1, wherein the controller is configured to iteratively initiate an inflation cycle and a deflation cycle during a testing duration defining a series of sub-durations, wherein at each subsequent sub-duration, the pressure in the inflatable member is adjusted.

10. An inflatable penile prosthesis comprising:
a fluid reservoir configured to hold fluid;
an inflatable member; and
an electronic pump assembly configured to transfer the fluid between the fluid reservoir and the inflatable member, the electronic pump assembly including:
an antenna configured to receive a wireless signal from an external device;
a pump;
an active valve;
a pressure sensor configured to measure a pressure of the inflatable member; and
a controller configured to control at least one of the pump or the active valve based on at least one of the measured pressure or the wireless signal.

11. The inflatable penile prosthesis of claim 10, wherein the electronic pump assembly includes an accelerometer configured to measure an acceleration of a user of the inflatable penile prosthesis, the controller configured to determine a type of activity based on the measured acceleration, wherein the controller is configured to adjust a pressure sensing rate associated with the pressure sensor based on the type of activity.

12. The inflatable penile prosthesis of claim 10, wherein the controller is configured to partially inflate the inflatable member such that the pressure of the inflatable member does not exceed a threshold, wherein, in response to the wireless signal, the controller is configured to inflate the inflatable member up to a maximum pressure threshold during an inflation cycle.

13. The inflatable penile prosthesis of claim 10, wherein the electronic pump assembly includes a temperature sensor configured to measure a temperature of the fluid, wherein, in response to the measured temperature being greater than a threshold, the controller is configured to transmit, over a network, a notification message via the antenna to one or more external devices.

14. The inflatable penile prosthesis of claim 10, wherein the controller includes a memory configured to store at least one of a maximum pressure threshold or a partial pressure threshold, the controller configured to update a value of at least one of the maximum pressure threshold or the partial pressure threshold based on information received from the external device via the antenna.

15. The inflatable penile prosthesis of claim 10, wherein the controller is configured to detect a performance issue associated with the inflatable penile prosthesis based on pressure readings from the pressure sensor, the controller configured to transmit, over a network, a notification message to one or more external devices in response to the performance issue being detected.

16. The inflatable penile prosthesis of claim 10, wherein the electronic pump assembly includes a battery configured to power the controller, the electronic pump assembly including a sensor configured to monitor a performance of the battery.

17. The inflatable penile prosthesis of claim 10, wherein the controller is configured to obtain pressure readings from the pressure sensor over time according to a pressure sensor rate, wherein the controller is configured to detect a first pressure pulsation at a first time and a second pressure pulsation at a second time, wherein the controller configured to cause the active valve to be in an open position after the first time, wherein the controller is configured to cause the active valve to be in a closed position before the second time.

18. The inflatable penile prosthesis of claim 10, wherein the electronic pump assembly includes a check valve in series with the pump.

19. A method of operating an inflatable penile prosthesis, the method comprising:
receiving, by an antenna of an electronic pump assembly, a wireless control signal from an external device;
activating, by a controller, a pump of the electronic pump assembly to operate to transfer fluid from a fluid reservoir to an inflatable member in response to the wireless control signal such that a pressure of the inflatable member reaches a threshold;
measuring, by a pressure sensor, the pressure of the inflatable member; and
activating, by the controller, an active valve of the electronic pump assembly to be in an open position to transfer a portion of the fluid from the inflatable member to the fluid reservoir in response to the measured pressure being greater than the threshold.

20. The method of claim 19, further comprising:
detecting, by the controller, that the measured pressure corresponds to the threshold; and
activating, by the controller, the active valve to be in a closed position in response to the measured pressure being detected as corresponding to the threshold.
